(19) United States
(12) Patent Application Publication
(10) Pub. No.: US 2008/0033465 A1
(43) Pub. Date: Feb. 7, 2008

(54) Multi-Wire Tissue Cutter

(75) Inventors: Gregory Schmitz, Los Gatos, CA (US); Jeffrerey Bleam, Boulder Creek, CA (US); Roy Leguidleguid, Union City, CA (US); Jeffery L. Bleich, Palo Alto, CA (US); Scott M. Smith, Redwood Shores, CA (US)

Correspondence Address: SHAYGLENN LLP, 2755 CAMPUS DRIVE, SUITE 210, SAN MATEO, CA, 94403, US

(73) Assignee: BAXANO, INC., Mountain View, CA (US)

(21) Appl. No.: 11/535,000

(22) Filed: Sep. 25, 2006

Related U.S. Application Data
(63) Continuation-in-part of application No. 11/461,740, filed on Aug. 1, 2006.

(51) Int. Cl. A61B 17/32

(52) U.S. Cl. 606/170

(57) ABSTRACT

A device for cutting tissue in a human body may include an elongate shaft having a proximal portion and a distal portion, at least one translatable blade disposed along one side of the distal portion of the shaft, and at least one actuator coupled with the at least one translatable blade and extending to the proximal portion of the shaft, wherein the actuator is configured to translate the blade to cut tissue. In various embodiments, various components of the device may have dimensions that facilitate passing a portion of the device into or through a small space and also facilitate and/or enhance the device's tissue cutting abilies.

### CROSS-REFERENCE TO RELATED APPLICATIONS

The present application is a continuation-in-part of U.S. patent application Ser. No. 11/461,740, entitled “Multi-Wire Tissue Cutter” (Attorney Docket No. 026445-000900US), and filed on Aug. 1, 2006, the disclosure of which is incorporated fully by reference.

### BACKGROUND OF THE INVENTION

The present invention relates generally to medical/surgical devices and methods. More specifically, the present invention relates to a tissue cutting devices and methods.

A significant number of surgical procedures involve cutting, shaving, abrading or otherwise contouring or modifying tissue in a patient's body. As the demand for less invasive surgical procedures continually increases, performing various tissue modifications such as cutting, contouring and removing tissue often becomes more challenging. Some of the challenges of minimally invasive procedures include working in a smaller operating field, working with smaller devices, and trying to operate with reduced or even no direct visualization of the structure (or structures) being treated. For example, using arthroscopic surgical techniques for repairing joints such as the knee or the shoulder, it may be quite challenging to cut certain tissues to achieve a desired result, due to the required small size of arthroscopic instruments, the confined surgical space of the joint, lack of direct visualization of the surgical space, and the like. It may be particularly challenging in some surgical procedures, for example, to cut or contour bone or ligamentous tissue with currently available minimally invasive tools and techniques. For example, trying to shave a thin slice of bone off a curved bony surface, using a small-diameter tool in a confined space with little or no ability to see the surface being cut, as may be required in some procedures, may be incredibly challenging or even impossible using currently available devices.

Examples of less invasive surgical procedures include laparoscopic procedures, arthroscopic procedures, and minimally invasive approaches to spinal surgery, such as a number of less invasive intervertebral disc removal, repair and replacement techniques. One area of spinal surgery in which a number of less invasive techniques have been developed is the treatment of spinal stenosis. Spinal stenosis occurs when one or more tissues in the spine impinges upon neural and/or neurovascular tissue, causing symptoms such as lower limb weakness, numbness and/or pain. This impingement of tissue may occur in one or more of several different areas in the spine, such as in the central spinal canal, or more commonly in the lateral recesses of the spinal canal and/or one or more intervertebral foramina.

FIGS. 1-3 show various partial views of the lower (lumbar) region of the spine. FIG. 1 shows an approximate top view of a vertebra with the cauda equina (the bundle of nerves that extends from the base of the spinal cord through the central spinal canal) shown in cross section and two nerve roots exiting the central spinal canal and extending through intervertebral foramina on either side of the vertebra. The spinal cord and cauda equina run vertically along the spine through the central spinal canal, while nerve roots branch off of the spinal cord and cauda equina between adjacent vertebrae and extend through the intervertebral foramina. Intervertebral foramina may also be seen in FIGS. 2 and 3, and nerves extending through the foramina may be seen in FIG. 2.

One common cause of spinal stenosis is buckling and thickening of the ligamentum flavum (one of the ligaments attached to and connecting the vertebrae), as shown in FIG. 1. (Normal ligamentum flavum is shown in cross section in FIG. 3) Buckling or thickening of the ligamentum flavum may impinge on one or more neurovascular structures, dorsal root ganglia, nerve roots and/or the spinal cord itself. Another common cause of neural and neurovascular impingement in the spine is hypertrophy of one or more facet joints (or “zygopophaseal joints”), which provide articulation between adjacent vertebrae. (Two vertebral facet superior articular processes are shown in FIG. 1. Each superior articular process articulates with an inferior articular process of an adjacent vertebra to form a zygopophaseal joint. Such a joint is labeled in FIG. 3.) Other causes of spinal stenosis include formation of osteophytes (or “bone spurs”) on vertebrae, spondylolisthesis (sliding of one vertebra relative to an adjacent vertebra), facet joint synovial cysts, and collapse, bulging or herniation of an intervertebral disc into the central spinal canal. Disc, bone, ligament or other tissue may impinge on the spinal cord, the cauda equina, branching spinal nerve roots and/or blood vessels in the spine to cause loss of function, ischemia and even permanent damage of neural or neurovascular tissue. In a patient, this may manifest as pain, impaired sensation and/or loss of strength or mobility.

In the United States, spinal stenosis occurs with an incidence of between 4% and 6% of adults aged 50 and older and is the most frequent reason cited for back surgery in patients aged 60 and older. Conservative approaches to the treatment of symptoms of spinal stenosis include systemic medications and physical therapy. Epidural steroid injections may also be utilized, but they do not provide long lasting benefits. When these approaches are inadequate, current treatment for spinal stenosis is generally limited to invasive surgical procedures to remove ligament, cartilage, bone spurs, synovial cysts, cartilage, and bone to provide increased room for neural and neurovascular tissue. The standard surgical procedure for spinal stenosis treatment includes laminectomy (complete removal of the lamina (see FIGS. 1 and 2) of one or more vertebrae) or laminotomy (partial removal of the lamina), followed by removal (or “resection”) of the ligamentum flavum. In addition, the surgery often includes partial or occasionally complete facetectomy (removal of all or part of one or more facet joints). In cases where a bulging intervertebral disc contributes to neural impingement, disc material may be removed surgically in a discectomy procedure.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. In a spinal fusion procedure, the vertebrae are attached together with some kind of support mechanism to prevent them from moving relative to one another and to allow adjacent vertebral bones to fuse together. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Discectomy procedures require entering through an incision in the patient's abdomen and navigating through the abdominal anatomy to arrive at the spine. Thus, while laminectomy, facetectomy, discectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients. Although a number of less invasive techniques and devices for spinal stenosis surgery have been developed, these techniques still typically require removal of significant amounts of vertebral bone and, thus, typically require spinal fusion.

Therefore, it would be desirable to have less invasive methods and devices for cutting, shaving, contouring or otherwise modifying target tissue in a spine to help ameliorate or treat spinal stenosis, while preventing unwanted effects on adjacent or nearby non-target tissues. Ideally, such techniques and devices would reduce neural and/or neurovascular impingement without removing significant amounts of vertebral bone, joint, or other spinal support structures, thereby avoiding the need for spinal fusion and, ideally, reducing the long-term morbidity levels resulting from currently available surgical treatments. It may also be advantageous to have tissue cutting devices capable of treating target tissues in parts of the body other than the spine, while preventing damage of non-target tissues. At least some of these objectives will be met by the present invention.

### SUMMARY OF THE INVENTION

In one aspect of the present invention, a device for cutting tissue in a human body may include an elongate shaft having a proximal portion and a distal portion, at least one translatable blade disposed along one side of the distal portion of the shaft, and at least one actuator configured to translate the blade to cut tissue coupled with the at least one translatable blade and extending to the proximal portion of the shaft. In some embodiments, the blade may have a height greater than a height of a portion of the shaft immediately below the blade, and a total height of the blade and the portion of the shaft immediately below the blade may be less than a width of the portion of the shaft immediately below the blade.

In some embodiments, the distal portion of the shaft may be sized to pass into an epidural space and at least partway into an intervertebral foramen of a spine. Optionally, the device may further include a backstop or a stationary blade toward which the translatable blade moves to cut tissue. In such embodiments, an edge of the backstop or stationary blade may be disposed at a blade opening distance from a cutting edge of the translatable blade. In some embodiments, the various components of the device may have a combination of dimensions. For example, in some embodiments, the blade opening distance may be between about 0.3 inches and about 0.35 inches, the height of the portion of the shaft immediately below the translatable blade may be between about 0.025 inches and about 0.035 inches, the height of the translatable blade may be between about 0.040 inches and about 0.060 inches, and the width of the portion of the shaft immediately below the blade may be between about 0.165 and about 0.250 inches. In some embodiments, a ratio of the height of the translatable blade to the height of the portion of the shaft immediately below the blade may be greater than or equal to one, or more preferably greater than or equal to about 4/3. In some embodiments, a ratio of the total height of the translatable blade and the height of the portion of the shaft immediately below the blade to the width of the portion of the shaft immediately below the blade may be less than or equal to one, or more preferably less than or equal to about ¾.

In some embodiments, the device may optionally include a guidewire coupling member disposed on the distal portion of the shaft for coupling the shaft with a guidewire to pull the device into a desired position and/or to apply tensioning force to the device to urge the translatable blade against target tissue. In some embodiments, the at least one actuator includes at least two flexible wires extending through a hollow lumen of the shaft to couple the actuator to the at least one translatable blade and a proximal actuation member coupled with the wires and the proximal portion of the shaft. In such embodiments, activating the actuation member may advance the wires to advance the blade along the shaft. In alternative embodiments, the at least one actuator may include at least one flexible wire extending through a hollow lumen of the shaft to couple the actuator to the at least one translatable blade and a proximal actuation member coupled with the wire(s) and the proximal portion of the shaft. In such embodiments, activating the actuation member may retract the wire(s) to retract the blade along the shaft.

Some embodiment of the device may optionally further include at least one chamber in or on the shaft for collecting cut tissue. In some embodiments, the shaft of the device may further include a flexible portion disposed between the proximal and distal portions, and the device may further include at least one shaft flexing actuator coupled with the proximal portion of the shaft and extending at least to the flexible portion of the shaft.

In another aspect of the present invention, a system for cutting tissue in a human body may include a tissue cutting device and a guidewire configured to couple with a guidewire coupling member of the tissue cutting device. The tissue cutting device may include: an elongate shaft having a proximal portion and a distal portion; at least one translatable blade disposed along one side of the distal portion of the shaft; at least one actuator coupled with the at least one translatable blade and extending to the proximal portion of the shaft, wherein the actuator is configured to translate the blade to cut tissue; and a guidewire coupling member disposed on the distal portion of the shaft for coupling the shaft with a guidewire to pull the device into a desired position and/or to apply tensioning force to the device to urge the translatable blade against target tissue. The blade of the tissue cutting device may have a height greater than a height of a portion of the shaft immediately below the blade, and a total height of the blade and the portion of the shaft immediately below the blade may be less than a width of the portion of the shaft immediately below the blade.

In some embodiments, the system may optionally further include a suction device and/or an irrigation device removably couplable with the tissue cutting device to provide at least one of suction and irrigation to the chamber to remove the cut tissue from the device. In such embodiments, the shaft of the tissue cutting device may further include at least one lumen for at least one of suction and irrigation. In some of the embodiments, the shaft of the device may further comprise a flexible portion disposed between the proximal and distal portions, and the device may further include at least one shaft flexing actuator coupled with the proximal portion of the shaft and extending at least to the flexible portion of the shaft. Optionally, the system may further include a guidewire handle for coupling with the guidewire outside the body to facilitate pulling the device into position and/or applying tensioning force.

These and other aspects and embodiments are described more fully below in the Detailed Description, with reference to the attached Drawings.

### BRIEF DESCRIPTION OF THE DRAWINGS

FIG. 1 is cross-sectional view of a spine, showing a top view of a lumbar vertebra, a cross-sectional view of the cauda equina, and two exiting nerve roots;

FIG. 2 is a left lateral view of the lumbar portion of a spine with sacrum and coccyx;

FIG. 3 is a left lateral view of a portion of the lumbar spine, showing only bone and ligament tissue and partially in cross section;

FIG. 4 is a cross-sectional view of a patient's back and spine with a side view of a tissue cutter device in place for performing a tissue removal procedure, according to one embodiment of the present invention;

FIG. 5A is side view of a tissue cutter device, showing blades of the device in an open position, according to one embodiment of the present invention;

FIG. 5B is a side view of the tissue cutter of FIG. 5A, showing the blades in a closed position;

FIG. 5C is a top view of a distal portion of the tissue cutter of FIGS. 5A and 5B, showing the blades in the open position;

FIG. 5D is a top view of the distal portion of FIG. 5C, with the blades in the closed position;

FIG. 5E is a side, cross-sectional view of a portion of the tissue cutter of FIGS. 5A-5D;

FIG. 5F is a magnified side view of the circled portion of the tissue cutter shown in FIG. 5E;

FIG. 5G is an end-on view of the portion of the tissue cutter shown in FIG. 5F, as seen from the direction labeled A in FIG. 5F;

FIG. 6 is a perspective view of a portion of a tissue cutter device, according to one embodiment of the present invention;

FIG. 7 is a perspective view of a window portion of a tissue cutter device, according to one embodiment of the present invention;

FIG. 8 is a perspective view of a window portion of a tissue cutter device, according to an alternative embodiment of the present invention;

FIGS. 9A-9F are side views of distal tips of various wires, according to various embodiments of the present invention;

FIGS. 10A-10G are end-on, cross-sectional views of various shafts and wire bundles of various tissue cutter devices, according to various embodiments of the present invention;

FIGS. 11A and 11B are side views of a distal portion of a tissue cutter device including a blade (FIG. 11A) and a bundle of wires (FIG. 11B), according to one embodiment of the present invention;

FIGS. 12A and 12B are side, cross-sectional views of a portion of a tissue cutter device including a ramping mechanism to urge one or more wires out of a window, according to one embodiment of the present invention;

FIG. 13 is a top view of a portion of a tissue cutter device including multiple wires and a radiofrequency wire cutter, according to one embodiment of the present invention;

FIG. 14 is a perspective view of a tissue cutter device including a squeeze handle and rigid and flexible shaft portions, according to one embodiment of the present invention;

FIG. 15 is a perspective view of a tissue cutter device including a rotary drive mechanism, according to one embodiment of the present invention; and

FIG. 16 is a perspective view of a tissue cutter device including an ultrasound drive mechanism, according to one embodiment of the present invention.

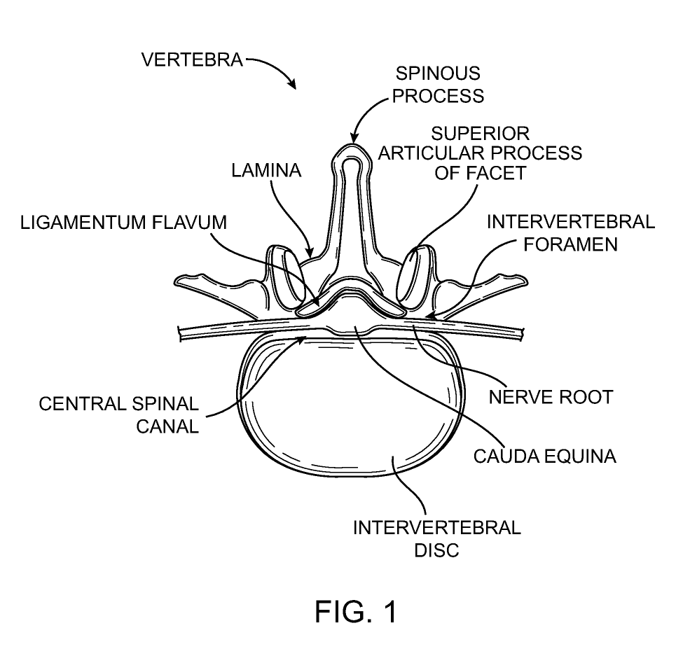

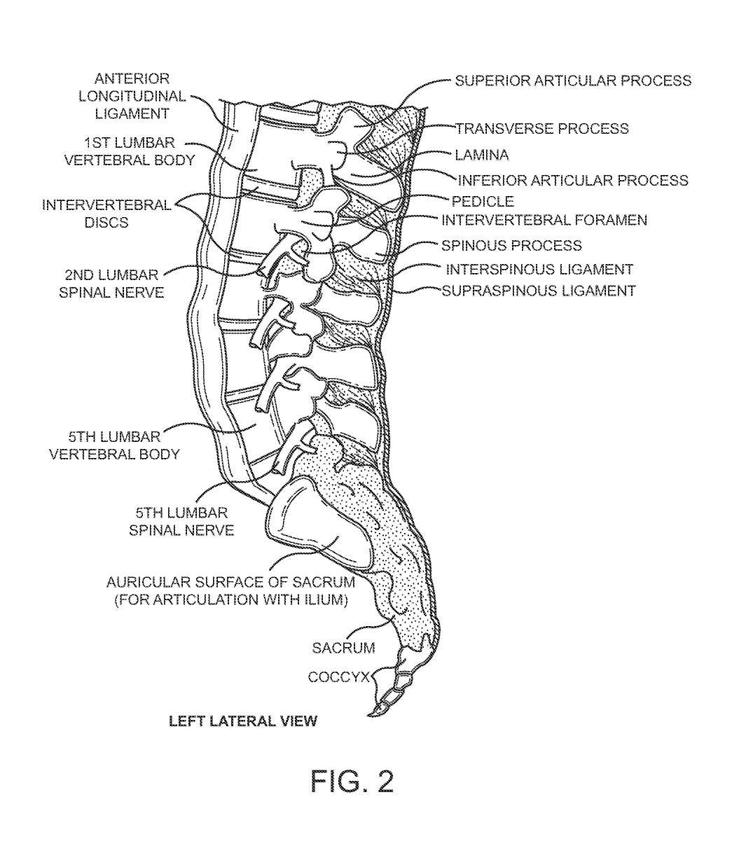

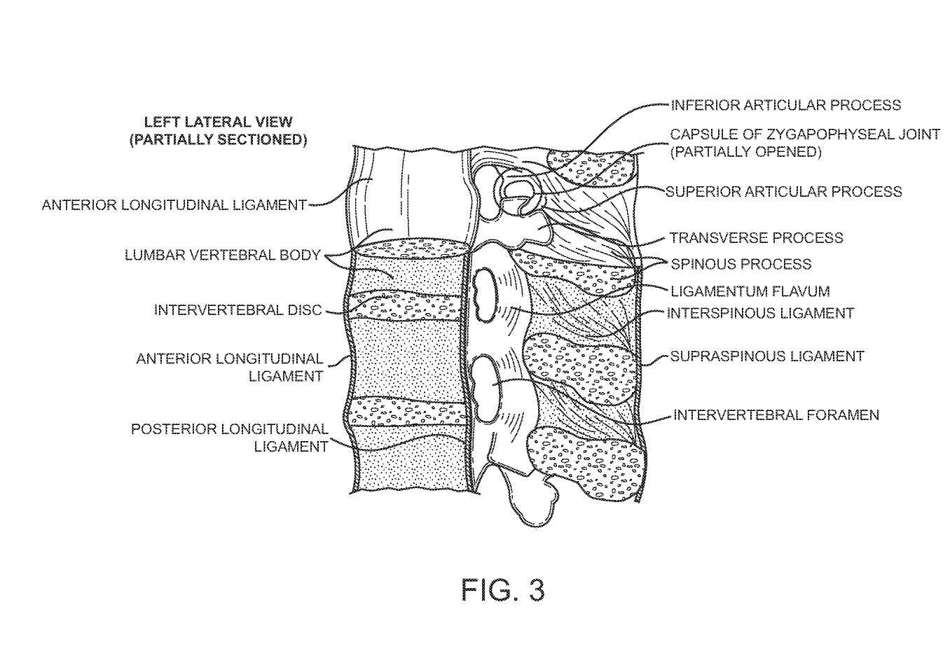

### DETAILED DESCRIPTION OF THE INVENTION

Various embodiments of a multiple-wire tissue cutter for modifying tissue in a patient are provided. Although the following description and accompanying drawing figures generally focus on cutting tissue in a spine, in various embodiments, any of a number of tissues in other anatomical locations in a patient may be modified.

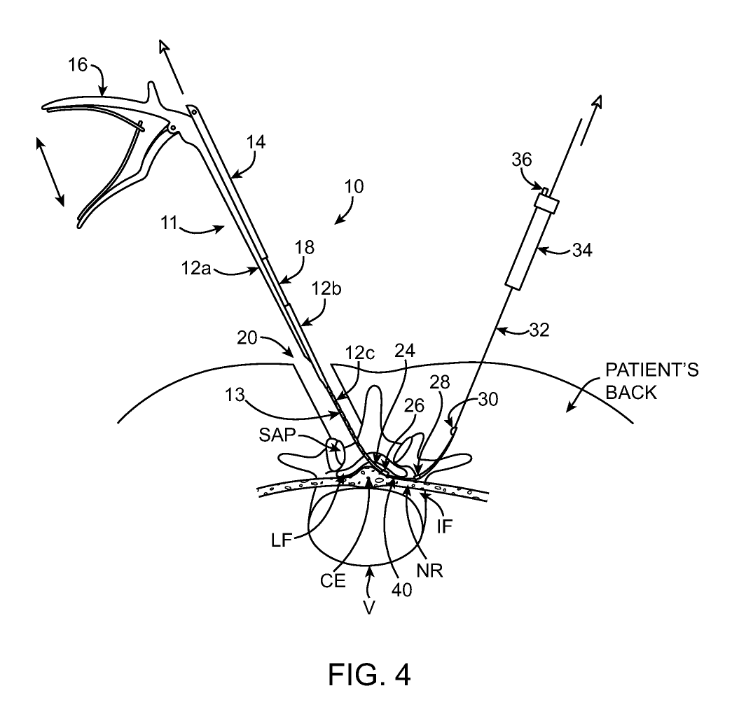

Referring to FIG. 4, one embodiment of a multi-wire tissue cutter device 10 may include a shaft having a proximal portion 11 and a distal portion 13. In some embodiments, proximal shaft portion 11 is predominantly rigid, and at least part of distal shaft portion 13 is flexible. Proximal shaft portion 11 may include a proximal stationary portion 12a coupled with or extending from a proximal handle 16, a distal stationary portion 12b, and a movable shaft portion 14. Distal shaft portion 13 may include a flexible shaft portion 12c and a platform 40 (also referred to herein as a “substrate,” “surface,” or “extension.”)

At least two flexible wires 24 (or “wire bundle”) may slidably extend through a portion of proximal shaft portion 11 and distal shaft portion 13 so that their distal ends attach to a proximal blade 26. Optionally, wires 24 may be bundled together along their entire lengths or along part of their lengths, and such a wire bundle may be partially housed within a wire bundle tube 18, which may slidably pass through distal stationary shaft portion 12b. Platform 40 may extend from shaft flexible portion 12c and may be coupled with a distal blade 28 and a guidewire connector 30. In various embodiments, part of platform 40, such as a portion immediately below blades 26, 28 and extending between blades 26, 28 may be relatively rigid, and part of platform 40, such as a portion distal to distal blade 28, may be relatively flexible. In some embodiments, tissue cutter device 10 (or a system including device 10) may further include additional features, such as a guidewire 32 configured to couple with guidewire connector 30 and a distal handle 34 (or “guidewire handle”) with a tightening lever 36 for coupling with guidewire 32.

In some embodiments, tissue cutter device 10 may be advanced into a patient's back through an incision 20, which is shown in FIG. 4 as an open incision but which may be a minimally invasive or less invasive incision in alternative embodiments. In some embodiments, device 10 may be advanced by coupling guidewire connector 30 with guidewire 32 that has been advanced between target and non-target tissues, and then pulling guidewire 32 to pull device 10 between the tissues. In alternative embodiments, device 10 may be advanced over guidewire 32, such as via a guidewire lumen or track. The flexibility of flexible portion 12c and at least part of the distal extension/platform may facilitate passage of device 10 between tissues in hard-to-reach or tortuous areas of the body, such as between a nerve root (NR) and facet joint and through an intervertebral foramen (IF). Generally, device 10 may be advanced to a position such that blades 26, 28 face tissue to be cut in a tissue removal procedure (“target tissue”) and one or more non-cutting surfaces of device 10 face non-target tissue, such as nerve and/or neurovascular tissue. In the embodiment shown in FIG. 4, blades 26, 28 are positioned to cut ligamentum flavum (LF) and may also cut hypertrophied bone of the facet joint, such as the superior articular process (SAP). (Other anatomical structures depicted in FIG. 4 include the vertebra (V) and cauda equina (CE)).

Before or after tissue cutter device 10 is pulled into the patient to pull blades 26, 28 to a desired position, guidewire 32 may be removably coupled with distal handle 34, such as by passing guidewire 32 through a central bore in handle 34 and tightening handle 34 around guidewire 32 via a tightening lever 36. Proximal handle 16 and distal handle 34 may then be pulled (hollow-tipped arrows) to apply tensioning force to device 10 and thus to urge the cutting portion of device 10 (e.g., blades 26, 28) against ligamentum flavum (LF), superior articular process (SAP), and/or other tissue to be cut. Proximal handle 16 may then be actuated, such as by squeezing in the embodiment shown (double-headed, solid-tipped arrow), which advances moveable shaft 14, thus advancing wire bundle tube 18, flexible wires 24 and proximal blade 26, to cut tissue between proximal blade 26 and distal blade 28. Proximal handle 16 may be released and squeezed as many times as desired to remove a desired amount of tissue. When a desired amount of tissue has been cut, guidewire 32 may be released from distal handle 34, and cutter device 10 and guidewire 32 may be removed from the patient's back.

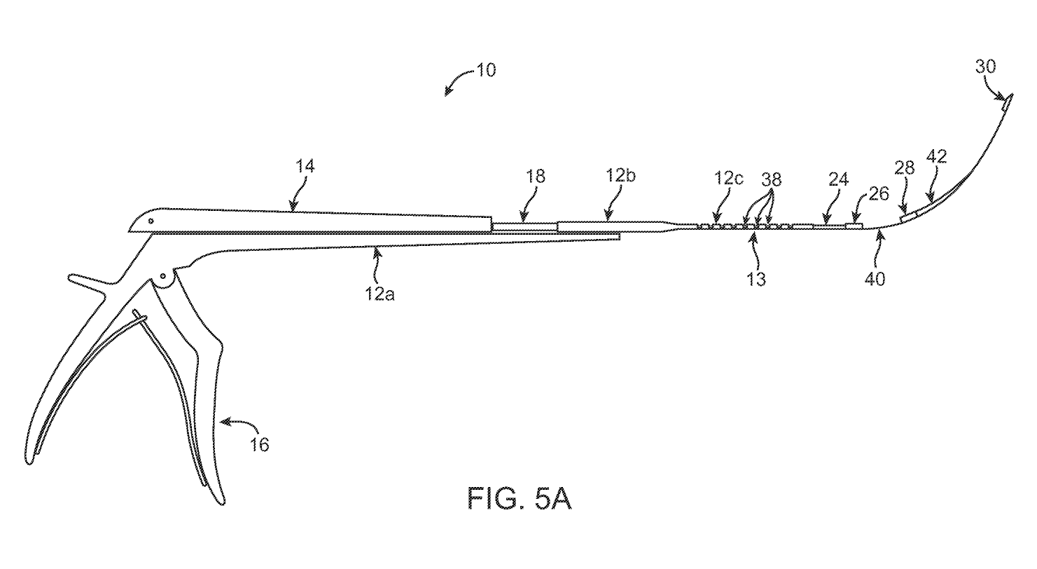

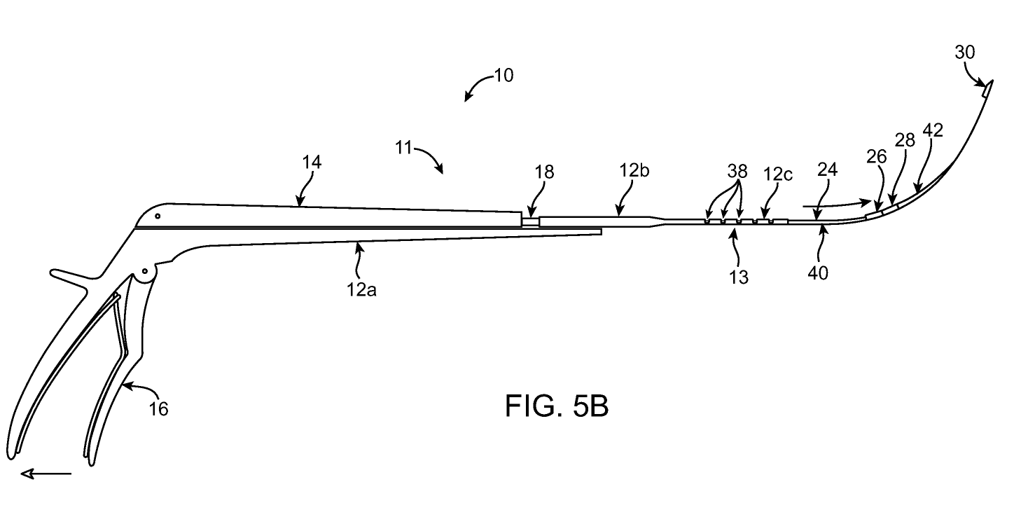

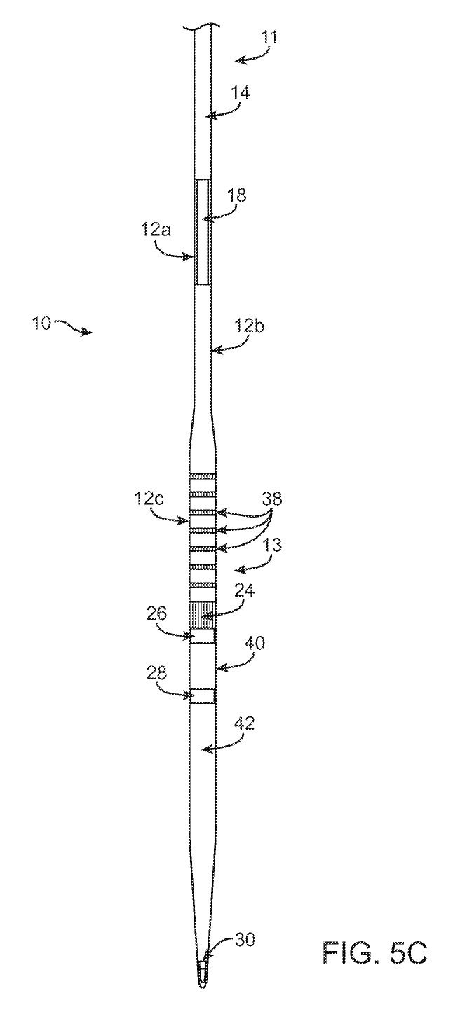

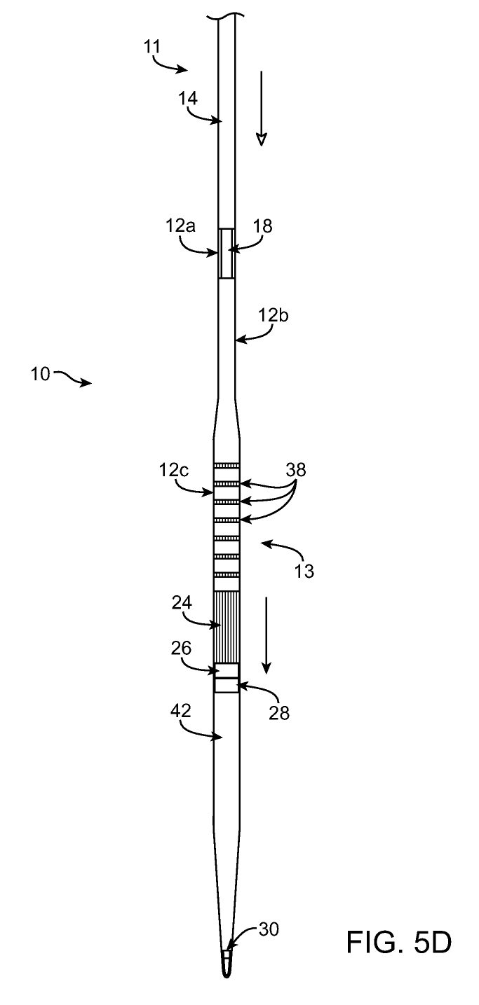

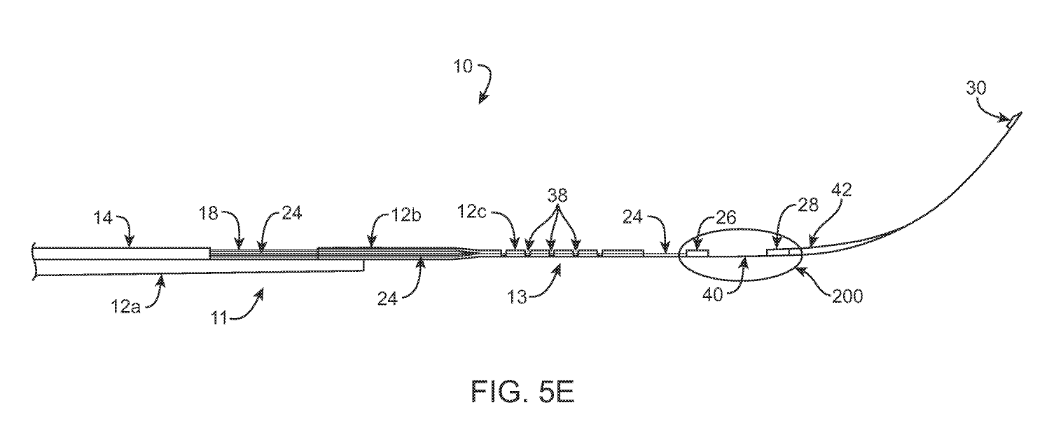

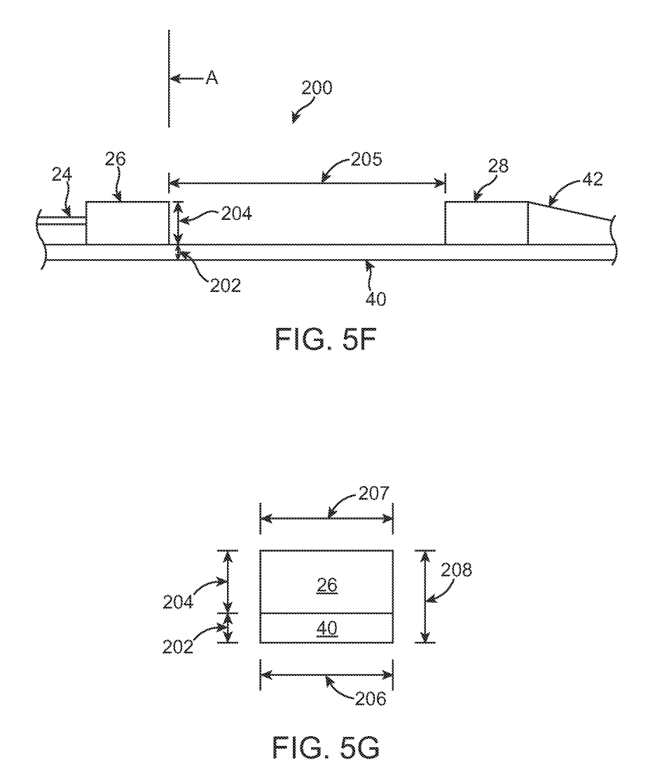

Referring now to FIGS. 5A-5G, tissue cutter device 10 of FIG. 4 is shown in greater detail. In FIG. 5A, a side view of cutter device 10 shows the device structure in greater detail. It can be seen, for example, that distal stationary shaft portion 12b tapers as it extends to flexible shaft portion 12c, which includes multiple slits 38 for enhancing flexibility. Generally, proximal shaft portion 11 and distal shaft portion 13 may be formed of any suitable material, such as but not limited to stainless steel. Wire bundle 24 extends through at least part of wire tube 18, through distal stationary shaft portion 12b and flexible shaft portion 12c, and is coupled with proximal blade 26. Wire tube 18 acts to secure the proximal end of wire bundle 24, such as by crimping, welding or the like. In alternative embodiments, wire tube 18 may be excluded, and the proximal end of wire bundle 24 may be otherwise coupled with device. For example, in various embodiments, wire bundle 24 may be coupled with moveable shaft portion 14, may be movably coupled with proximal handle 16, or the like. Extending distally from flexible shaft portion 12c is a platform 40 (or “substrate,” “surface” or “extension”), on which are mounted distal blade 28, a tissue collection chamber 42 and guidewire connector 30. (For the purposes of this application, in various embodiments, the various parts of shaft 12, 14 and platform 40 may be referred to together as the “body” of device 10 or a “device body.”) Platform 40 generally extends underneath proximal blade 26, between blades 26, 28, and underneath distal blade 28. In some embodiments, platform 40 may be rigid, while in alternative embodiments, platform may be flexible. Collection chamber 42 may be a hollow chamber continuous with distal blade 28, configured such that cut tissue may pass under distal blade 28, into chamber 42. In the side view of FIG. 5A, wire bundle 24 appears as a single wire, in this embodiment due to the fact that flattened flexible portion 12c flattens wire bundle 24 to a one-wire-thick cross section. In FIG. 5A, blades 26, 28 are shown in the open position.

In various embodiments, proximal shaft portion 11 and distal shaft portion 13 may have any suitable shapes and dimensions and may be made of any suitable materials. For example, in various embodiments, shaft portions 11, 13 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides.

Portions of shaft 11, 13 through which wire bundle 24 travels will generally be predominantly hollow, while other portions may be either hollow or solid. For example, in one embodiment, moveable shaft portion 14 and proximal stationary portion 12a may be solid, distal stationary portion 12b and flexible shaft portion 12c may be hollow, and platform 40 may be a flat piece of material. Although one particular embodiment of a shaft mechanism for moving wire bundle 24 is shown, various embodiments may employ any of a number of alternative mechanisms. For example, one embodiment may include a largely or completely flexible shaft, such as an elongate catheter shaft, which extends directly from proximal handle 16. In such an embodiment, wire bundle 24 may couple directly with a drive mechanism of handle 16, so that handle 16 reciprocates wire bundle 24 without employing a rigid shaft structure. In another embodiment, moveable shaft portion 14 may be at least partially hollow, and wire bundle 24 may extend into moveable portion 14 and be attached therein. Therefore, the embodiment of device 10 in FIGS. 4 and 5A-5G is but one example of a multi-wire tissue cutter device. In various alternative embodiments, any of a number of changes may be made to the structure of device 10.

As mentioned above, the various components of shaft proximal and distal portions 11, 13 may have any of a number of shapes. For example, the hollow portions of shaft 12b and 12c, through which wire bundle 24 passes, may have any of a number of cross-sectional shapes in various embodiments. As shown in FIGS. 5A-5E, for example, distal rigid portion 12b may have a round cross-sectional shape, and flexible portion 12c may have a flat shape. In other embodiments, hollow portions 12b, 12c may have one or more other cross-sectional shapes, such as but not limited to round, ovoid, ellipsoid, flat, cambered flat, rectangular, square, triangular, symmetric or asymmetric cross-sectional shapes. In another alternative embodiment, a hollow portion of a shaft may have a continuous cross-sectional shape along its entire length. In some embodiments, at least the distal shaft portion 13 may have a small profile, to facilitate passage of that portion into a patient, through an introducer device, between target and non-target tissues, through one or more small anatomical channels and/or around an anatomical curve with a small radius of curvature. In some embodiments, for example, distal shaft portion 13 may have a height of not more than about 10 mm at any point along its length and a width of not more than about 20 mm at any point along its length, or more preferably a height not more than about 5 mm at any point along its length and a width of not more than about 10 mm at any point along its length, or even more preferably a height not more than about 2 mm at any point along its length and a width of not more than about 4 mm at any point along its length. Dimensions of various portions and embodiments of device 10 are discussed further below, in reference to FIGS. 5F and 5G.

Shaft flexible portion 12c generally has a configuration and thickness to provide some amount of flexibility, and its flexibility may be further enhanced by one or more slits 38 in an upper surface of the shaft material. Any number and width of slits 38 may be used, in various embodiments, to confer a desired amount of flexibility. In various embodiments, for example, anywhere from one to 100 slits may be formed in the upper surface of flexible shaft portion 12c. In some embodiments, slits may have varying widths and/or may be placed at varying distances from one another, to provide more flexibility along one or more sections of flexible shaft portion 12c and less flexibility along other sections.

In various embodiments, platform 40 may comprise an extension of a lower surface of shaft flexible portion 12c. Alternatively or additionally, platform 40 may comprise one or more separate pieces of material coupled with shaft flexible portion 12c, such as by welding or attaching with adhesive. Platform 40 may comprise the same or different material(s) as shaft 12, according to various embodiments, and may have any of a number of configurations. For example, platform 40 may comprise a flat, thin, flexible strip of material (such as stainless steel), as shown in FIG. 5A. In an alternative embodiment, platform 40 may have edges that are rounded up to form a track through which proximal blade 26 may travel. In some embodiments, platform 40 may be flexible, allowing it to bend, while in other embodiments, platform 40 may be predominantly rigid, so that it does not bend or bends only slightly when device 10 is placed under tension around a curved surface. In various embodiments, platform 40 may be made more rigid by making platform 40 more think and/or by using more rigid material to construct platform 40. In some embodiments, platform 40 may be made of a shape memory material and given a curved shape, while in other embodiments platform 40 may be rigid and curved or rigid and straight. Differently shaped platforms 40 and/or platforms 40 having different amounts of flexibility may facilitate use of different embodiments of tissue cutter device 10 in different locations of the body. A more rigid platform 40, for example, may facilitate cutting of a hard material such as bone with blades 26, 28.

Some embodiments of device 10 may further include one or more electrodes coupled with platform 40 and/or flexible shaft portion 12c, for transmitting energy to tissues and thereby confirm placement of device 10 between target and non-target tissues. For example, electrodes may be placed on a lower surface of platform 40 and/or an upper surface of flexible shaft portion 12c, and the electrodes may be separately stimulated to help confirm the location of neural tissue relative to blades 26, 28. In such embodiments, nerve stimulation may be observed as visible and/or tactile muscle twitch and/or by electromyography (EMG) monitoring or other nerve activity monitoring. In various alternative embodiments, additional or alternative devices for helping position, use or assess the effect of tissue cutter device 10 may be included. Examples of other such devices may include one or more neural stimulation electrodes with EMG or SSEP monitoring, ultrasound imaging transducers external or internal to the patient, a computed tomography (CT) scanner, a magnetic resonance imaging (MRI) scanner, a reflectance spectrophotometry device, and a tissue impedance monitor disposed across a bipolar electrode tissue modification member or disposed elsewhere on tissue cutter device 10.

Wire bundle 24 may include as few as two flexible wires 24 and as many as one hundred or more wires 24. In some embodiments, for example, between three and 20 wires 24 may be used, and even more preferably, between four and ten wires 24. Wires 24 may have any of a number of different diameters, so in some embodiments the number of wires 24 used may be determined by the diameter of wire 24 used. In various embodiments, each wire 24 may be a solid wire, a braided wire, a core with an outer covering or the like, and may be made of any suitable material. For example, in various embodiments, wires 24 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for the wires 24 or for portions or coatings of the wires may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In some embodiments, all wires 24 may be made of the same material, whereas in alternative embodiments, wires 24 may be made of different materials. Individual wires 24 may also have any length, diameter, tensile strength or combination of other characteristics and features, according to various embodiments, some of which are discussed in greater detail below.

In various embodiments, flexible wires 24 may be bound or otherwise coupled together at one or more coupling points or along the entire length of wire bundle 24. In one embodiment, for example, wires 24 may be coupled together by a sleeve or coating overlaying wire bundle 24. In another embodiment, wires 24 may only be coupled together at or near their proximal ends, at or near their connection point to tube 18, moveable shaft portion 14 or the like. In an alternative embodiment, wires 24 may be individually coupled with an actuator, such as proximal handle 16, and not coupled to one another directly. In any case, wires 24 will typically be able to move at least somewhat, such as laterally, relative to one another. This freedom of movement facilitates the change of cross-sectional shape that wire bundle 24 undergoes as it passes through differently shaped hollow portions of shaft 12b, 12c. The change in cross-sectional shape of wire bundle 24 may convey different properties on device 10 at different portions, such as enhanced rigidity at one portion and enhanced flexibility at another.

In some embodiments, wires 24 may be individually coupled with a proximal actuator and may also be bound together at least one point along their lengths. Optionally, such a proximal actuator may allow one or more individual wires to be pulled, pushed and/or twisted, which acts to steer wire bundle 24 and thus steer a distal portion of device 10. In alternative embodiments, one or more wires 24 or other mechanisms, separate from wire bundle 24, may be used to steer distal shaft portion 13. In some embodiments, for example, proximal shaft portion 11 and distal shaft portion 13 may both be rigid, and device 10 may further include a flexible portion between the two. One or more tensioning wires may extend from proximal handle 16, where they may be coupled with an actuator, to at least the flexible portion of the shaft and in some embodiments to the rigid distal shaft portion 13. The tensioning wire may be pulled or tensioned to bend the flexible portion, thus articulating distal portion 13. In another embodiment, one or more compressive wires or other compressive mechanism(s) may be used to apply compressive force to bend the flexible portion of shaft and articulate distal portion 13. A number of suitable shaft steering mechanisms and techniques may be applied, according to various embodiments.

In some embodiments, wire bundle 24 may include one or more elongate, flexible members for performing various functions, such as enhancing tissue cutting, visualizing a target area or the like. For example, in various embodiments, bundle 24 may include one or more optical fibers, flexible irrigation/suction tubes, flexible high pressure tubes, flexible insulated tubing for carrying high temperature liquids, flexible insulated tubing for carrying low temperature liquids, flexible elements for transmission of thermal energy, flexible insulated wires for the transmission of electrical signals from a sensor, flexible insulated wires for the transmission of electrical signals towards the distal end of the wires, energy transmission wires, or some combination thereof. Examples of visualization devices that may be used include flexible fiber optic scopes, CCD (charge-coupled device) or CMOS (complementary metal-oxide semiconductor) chips at the distal end of flexible probes, LED illumination, fibers or transmission of an external light source for illumination or the like.

When blades 26, 28 face target tissue to be modified, such as buckled, thickened or otherwise impinging ligamentum flavum tissue, device 10 is configured such that platform 40 faces non-target tissue. Platform 40 may thus act as a tissue protective surface, and in various embodiments platform 40 may have one or more protective features, such as a width greater than the width of blades 26, 28, rounded edges, bumpers made of a different material such as a polymer, protective or lubricious coating(s), extendable or expandable barrier member(s), drug-eluting coating or ports, or the like. In some instances, platform 40 may act as a “non-tissue-modifying” surface, in that it may not substantially modify the non-target tissue. In alternative embodiments, platform 40 may affect non-target tissue by protecting it in some active way, such as by administering one or more protective drugs, applying one or more forms of energy, providing a physical barrier, or the like.

Generally, blades 26, 28 may be disposed on platform 40. Proximal blade 26 may be unattached or moveably/slidably attached to platform 40, so that it is free to translate (or “reciprocate”) along platform 40 with the back and forth movement of wire bundle 24. In one embodiment, for example, proximal blade 26 may be slidably coupled with platform 40 via a piece of material wrapped around blade 26 and platform 40. In another embodiment, proximal blade 26 may slide through one or more tracks on platform 40. Distal blade 28 may be fixedly attached to platform 40 and thus remain stationary, relative to platform 40, such that proximal blade 26 translates toward stationary distal blade 28 to cut tissue. In alternative embodiments, the distal end of wire bundle 24, itself, may be used to cut tissue, and device 10 may thus not include proximal blade 26. For example, each wire 24 may have a sharp, tissue cutting point, or wire bundle 24 as a whole may form a sharp, tissue cutting edge. The distal end of wire bundle 24 may advance toward distal blade 28 to cut target tissue, or in alternative embodiments, wire bundle 24 may advance toward a non-sharp backstop to cut tissue or may simply advance against tissue to ablate it, without pinching the tissue between the wire bundle 24 distal end and any other structure. An example of the latter of these embodiments might be where ultrasound energy is used to reciprocate wire bundle 24, in which case the reciprocation of wire bundle 24 may be sufficient to cut or ablate tissue, without pinching or snipping between wire bundle and another structure.

In various embodiments, blades 26, 28, or other cutting structures such as the distal ends of wire bundle 24, a backstop or the like, may be disposed along any suitable length of distal shaft portion 13 and/or platform 40. In the embodiment shown in FIG. 5A, for example, blades 26, 28 are disposed along a length of platform 40. In an alternative embodiment, distal shaft portion 13 may comprise a hollow portion through which wire bundle 24 travels and a window through which wire bundle 24 is exposed. In any case, blades 26, 28 or other cutting members may be disposed or exposed along a desired length of device 10, to help limit an area in which the cutting members are active, thus helping to limit the exposure of non-target tissues to such cutting elements. In one embodiment, for example, such as an embodiment of the device to be used in a spinal treatment, blades 26, 28 may be disposed along a length of platform 40 measuring no longer than about 10 cm, and preferably no more than about 6 cm, and even more preferably no more than about 3 cm. In various embodiments, the length along which blades 26, 28 are disposed may be selected to approximate a length of a specific anatomical treatment area.

Blades 26, 28 may be made from any suitable metal, polymer, ceramic, or combination thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for blades 26, 28 or for portions or coatings of blades 26, 28 may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In various embodiments, blades 26, 28 may be manufactured using metal injection molding (MIM), CNC machining, injection molding, grinding and/or the like. Proximal and distal blades 26, 28 may be attached to wire bundle 24 and platform 40, respectively, via any suitable technique, such as by welding, adhesive or the like.

Tissue collection chamber 42 may be made of any suitable material, such as but not limited to any of the materials listed above for making blades 26, 28. In one embodiment, for example, chamber 42 may comprise a layer of polymeric material attached between distal blade 28 and platform 40. In another embodiment, collection chamber 42 and distal blade 28 may comprise one continuous piece of material, such as stainless steel. Generally, distal blade 28 and chamber 42 form a hollow, continuous space into which at least a portion of cut tissue may pass after it is cut.

Guidewire connector 30 generally comprises a member build into or coupled with platform 40, at or near its distal tip, for coupling device 10 with a guidewire. In some embodiments, for example, guidewire connector 30 may be formed from the same piece of material that forms platform 40. For example, connector 30 may include a receptacle for accepting a shaped tip (ball, cylinder or the like) of a guidewire and holding it to prevent unwanted guidewire release. A number of such guidewire connectors 30 and guidewires are described in U.S. patent Ser. Nos. 11/468,247 and 11/468,525 (Attorney Docket Nos. 026445-001000US and 026445-001100US, respectively), both of which are titled “Tissue Access Guidewire System and Method,” and both of which were filed on Aug. 29, 2006, the full disclosures of which are hereby incorporated by reference. In alternative embodiments, connector 30 may be replaced with a guidewire lumen or track for advancing device 10 over a guidewire.

With reference now to FIG. 5B, proximal handle 16 may be squeezed (hollow-tipped arrow) to advance moveable shaft portion 14, which thus pushes against wire bundle tube 18 to advance wire bundle 24 and proximal blade 26 (solid-tipped arrow). Handle 16 may then be released and squeezed again as many times as desired to cut a desired amount of tissue.

The advancement of proximal blade 26 is also depicted in FIGS. 5C and 5D. FIG. 5C is a top view of a portion of tissue cutter device 10, showing the multiple flexible wires 24 of wire bundle 24 and showing blades 26, 28 in the open position. FIG. 5D shows the moveable shaft portion 14 advanced (hollow-tipped arrow) and wire bundle 24 and proximal blade 26 advanced to meet distal blade 28.

Referring to FIG. 5E, a cross-sectional view of a portion of device 10 demonstrates that wire bundle 24 assumes the cross-sectional shape of distal stationary shaft portion 12b where it is disposed in that portion and assumes the cross-sectional shape of flat flexible portion 12c where it is disposed in that portion. Thus, in some embodiments, wire bundle 24 may assume the cross-sectional shape of the shaft or other containing structure in which it resides. In other words, as wire bundle 24 translates through the differently shaped hollow shaft portions 12b, 12c, its cross-sectional shape changes along at least a portion of its length to assume approximately the shape of the shaft portion containing it.

Referring now to FIGS. 5F and 5G, a side view (FIG. 5F) and an end-on view (FIG. 5G) of a portion 200 of device 10 (circled in FIG. 5E) are shown. (FIG. 5F is a view from the perspective labeled A in FIG. 5F.) It has been found that in some embodiments, various components and portions of tissue cutting device 10 may preferably have a combination of dimensions that facilitate passage into a small space and effective tissue cutting. In various embodiments, the dimensions described below may be applied to any tissue cutting device, especially devices designed to cut tissue located in small anatomical passageways or spaces, such as in and around an intervertebral foramen of a spine. In other words, although tissue cutter device 10 has generally been described as multi-wire tissue cutter 10 in the present application, the dimensions and combinations of dimensions described below may be applied to other tissue cutting devices, without departing from the scope of the present invention. For example, a number of alternative tissue cutting devices are described in U.S. patent application Ser. No. 11/405,848, entitled “Mechanical Tissue Modification Devices and Methods” (Original Attorney Docket No. 78117-200301), and filed Apr. 17, 2006, the full disclosure of which is hereby incorporated by reference. In that disclosure, for example, one of the embodiments a tissue cutting device includes a translatable blade that is retracted via two pull wires. It is contemplated that the dimensional characteristics described below may be applied to such a device, as well as to other tissue cutting devices in other alternative embodiments.

Referring again to FIGS. 5F and 5G, in one embodiment, platform 40 (or “substrate”) may have a substrate height 202 (or “thickness”), blades 26, 28 may have a blade height 204, edges of blades 26, 28 may be separated by a blade opening distance 205, blades 26, 28 may have a blade width 207, platform 40 may have a substrate width 206, and each blade 26, 28 together with platform 40 may have a total device height 208. (Substrate height 202 or substrate width 206 may also be referred to as the height or width of “a portion of the shaft immediately below the blade(s).”) Each of these various dimensions may be adjusted according to various embodiments and for various applications to different parts of patient anatomy. Some embodiments, for example, may be configured for use in and near an intervertebral foramen of a spine. In an alternative embodiment, dimensions of device 10 may be selected for use in a shoulder surgery procedure, a knee surgery procedure, a hand surgery procedure or the like.

In some embodiments, the portion 200 of device 10 may have an overall size and dimensions such that it may be passed into an epidural space of a spine and at least partially into an intervertebral space of the spine, so that it may be used to cut ligament and/or bone in the spine to treat neural and/or neurovascular impingement. In some embodiments, for example, substrate height 202 may be less than or equal to blade height 204. In other words, the ratio of substrate height 202 to blade height may be approximately less than or equal to one, and in some embodiments approximately less than or equal to ¾. In these or other embodiments, total height 208 (of blade 26 and platform 40) may be less than or equal to substrate width 206 and/or blade width 207. (In some embodiments, substrate width 206 may be approximately equal to blade width 207, as shown, while in alternative embodiments, substrate width 206 may be greater than blade width 207.) In other words, the ratio of total height 208 to width 207 may be approximately less than or equal to one, and in some embodiments approximately less than or equal to ¾. In some embodiments, device 10 may have a combination of a ratio of substrate height 202 to blade height approximately less than or equal to one and a ratio of total height 208 to width 206 approximately less than or equal to one. Such a configuration is contrary to that of traditional rongeurs, which include cutting blades thinner than their underlying supporting structure and which have a total height greater than the width of the device. In one embodiment, for example, blade opening distance 205 may be between about 0.1 inches and about 0.5 inches, substrate height 202 may be between about 0.010 inches and about 0.050 inches, blade height 204 may be between about 0.010 inches and about 0.075 inches, and blade width 207 may be between about 0.130 and about 0.400 inches. More preferably, in one embodiment, blade opening distance 205 may be between about 0.3 inches and about 0.35 inches, substrate height 202 may be between about 0.025 inches and about 0.035 inches, blade height 204 may be between about 0.040 inches and about 0.060 inches, and blade width 207 may be between about 0.165 and about 0.250 inches. In alternative embodiments, such as for use in other parts of the body, device 10 may have any of a number of different combinations of dimensions.

To optimize tissue cutter device 10 for any of a number of possible uses, the dimensions described above may be combined with any of a number of materials for the various components of device 10. Examples of such materials for blades 26, 28, platform 40 and the like have been listed previously. In some embodiments, for example, platform 40 may be made of a material and may have a height or thickness 202 such that it is predominantly stiff or rigid, even when placed under tension against a rounded surface. In another embodiment, platform 40 may be more flexible, to allow for greater bending around a surface. Using various combinations of dimensions and materials, device 10 may be configured to cut any of a number of tissues in any of a number of locations in the body.

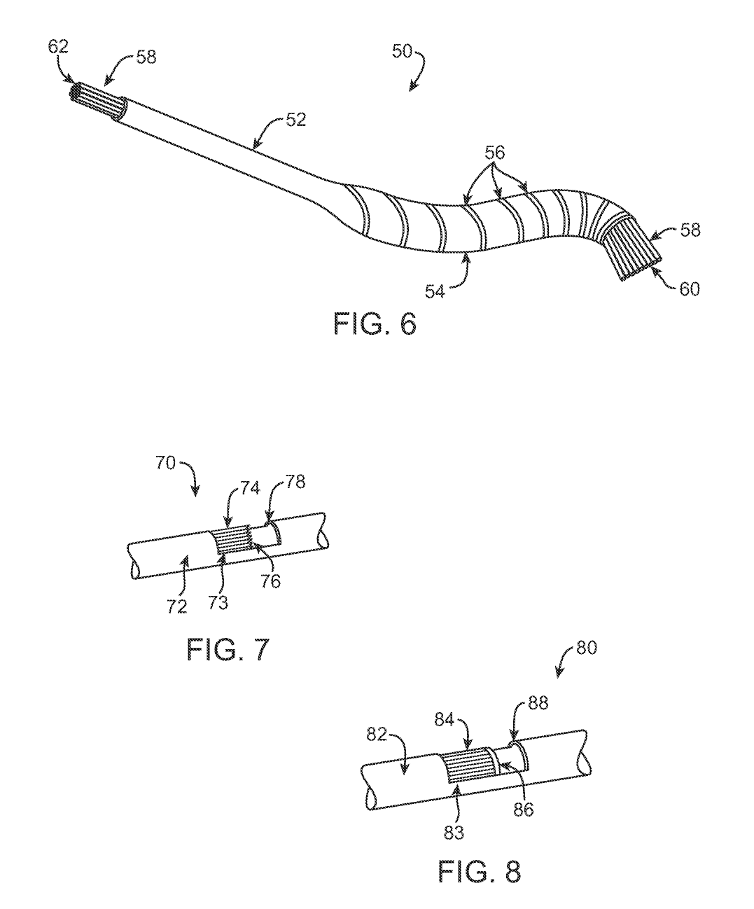

With reference now to FIG. 6, a portion of a tissue cutter device 50 is shown, in this embodiment including proximal shaft portion 52, a distal shaft portion 54 having multiple slits 56, and a wire bundle 58 disposed within shaft 52, 54. Each wire of bundle 58 includes a distal end 60 and a proximal end 62. This portion of device 50 shows in greater detail how in some embodiments wire bundle 58 may have a first cross-sectional configuration in one portion of shaft 52 and a second cross-sectional configuration in another portion of shaft 54. In fact, the cross-sectional shape of a portion of bundle 58 may change as that portion passes from proximal shaft portion 52 to distal shaft portion 54 or vice versa. Changing the cross-sectional shape of wire bundle 58 along the length of shaft 52, 54 may enhance flexibility of device 50 along one or more portions and/or may give one or more portions of device 50 an overall shape that facilitates its passage between closely apposed tissues, through a small channel, around a tight corner or the like. Wire bundle 58 will be disposed within shaft 52, 54 such that the individual wires of the bundle have at least some freedom to move relative to one another, thus enabling the cross-sectional shape of bundle 58 to change. In various alternative embodiments, wire bundle 58 may have any of a number of cross-sectional shapes, and may either change from one shape to another as it passes through shaft 52, 54 or, alternatively, may maintain the same shape throughout the length of an alternative shaft. As has been mentioned previously, further flexibility may be conferred on device 50 via slits 56.

In some embodiments, the changeability of the cross-sectional shape of wire bundle 58 may also be used to measure a contour or shape of an anatomical structure. For example, flexible bundle of wires 58 may be pressed against a contour to be measured, and bundle 58 may then be locked, to lock the cross-sectional shape of the contour into bundle 58. Device 50 may then be withdrawn from the patient, and the contour measured or otherwise assessed.

In some embodiments, rather than coupling the distal end of wire bundle 58 with a blade, distal ends 60 of the wires themselves may be used to cut tissue. Distal tips 60 may have any of a number of configurations, some of which are described in greater detail below. These ends 60 may be used to cut, scrape, pummel, chisel, shatter, ablate or otherwise modify tissue in various embodiments. In some embodiments, wire bundle 58 may be advanced and retracted using a manually powered handle to cut tissue with ends 60. Alternatively, as will be described further below, ends 60 may be reciprocated using ultrasound energy, using a rotational, powered driving mechanism, or the like.

Referring to FIG. 7, a portion of an alternative embodiment of a tissue cutter device 70 may include a shaft 72 with a window 73 and a wire bundle 74 slidably disposed within shaft 72. The individual wires of bundle 74 may include distal tips 76, which may be sharpened in some embodiments. Wire bundle 74 may be reciprocated back and forth to cut tissue through window 73. In some embodiments, window 73 may include a sharpened edge 78, and tips 76 of wire bundle 74 may work with edge 78 to cut or snip off tissue. In an alternative embodiment, sharpened edge 78 may be left off, and distal tips 76 may advance tissue against a blunt or rounded edge of window 73.

As is evident from FIG. 7, in some embodiments, shaft 72 and wire bundle 74 may have a generally round cross-sectional shape. Such a configuration may be advantageous, for example, if shaft 72 is a flexible, elongate catheter. In some embodiments, the individual wires of wire bundle 74 may be free enough to move, relative to one another, that they can conform to a surface to be cut, such as a curved surface of a bone or the like. Such a shape conformation may facilitate even cutting of a tissue surface.

In an alternative embodiment, and with reference now to FIG. 8, a tissue cutter device 80 may include a shaft 82 with a window 83, a wire bundle 84 slidably disposed within shaft 82, a curved blade 86 coupled with the distal end of bundle 84, and a sharpened edge 88 of window 83. In an alternative embodiment, sharpened edge 88 may be left off, and blade 86 may advance tissue against a blunt or rounded edge of window 83.

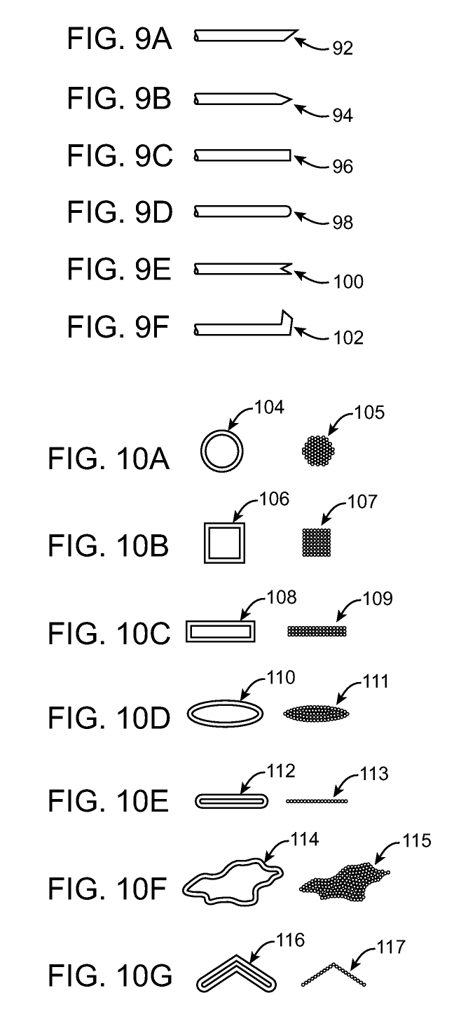

FIGS. 9A-9F show distal ends (or “tips”) of a variety of wires, which may be used to form wire bundles according to various embodiments of the tissue cutters described herein. These figures are provided for exemplary purposes only, and other embodiments of wires may have alternative shapes. In the embodiments shown, a wire may have a beveled tip 92 (FIG. 9A), double-beveled tip 94 (FIG. 9B), flat/squared-off tip 96 (FIG. 9C), rounded tip 98 (FIG. 9D), inverted double-bevel tip 100 (FIG. 9E), or bent/scraper tip 102 (FIG. 9F). Additionally, various wires may have any desired diameter, length, tensile strength or cross-sectional shape. For example, a typical wire may have a round cross-sectional shape, but alternative wires may have oval, square, rectangular, triangular, hexagonal or other cross-sectional shapes.

Referring now to FIGS. 10A-10G, just as wires may have different tip shapes in different embodiments, shafts and wire bundles may have different cross-sectional shapes in different embodiments. Typically, the cross-sectional shape of a shaft will determine the cross-sectional shape of a wire bundle that passes through it, since the wires of the bundle will be at least somewhat free, relative to one another. As has been described above, in various embodiments, a shaft may have one cross-sectional shape along its entire length or, alternatively, it may have two or more different cross-sectional shapes, such as a round shape proximally and a flatter shape distally. The embodiments shown, which are merely examples, include a round shaft 104 with a round wire bundle 105 (FIG. 10A), a square shaft 106 with a square wire bundle 107 (FIG. 10B), a rectangular shaft 108 with a rectangular wire bundle 109 (FIG. 10C), an oval shaft 110 with an oval wire bundle 111 (FIG. 10D), a flat shaft 112 with a flat wire bundle 113 (FIG. 10E), an asymmetric shaft 114 with an asymmetric wire bundle 115 (FIG. 10F), and a V-shaped shaft 116 with a V-shaped wire bundle 117 (FIG. 10G). Any of these shapes or other shapes may be used alone or in combination in any given embodiment of a multi-wire tissue cutter device.

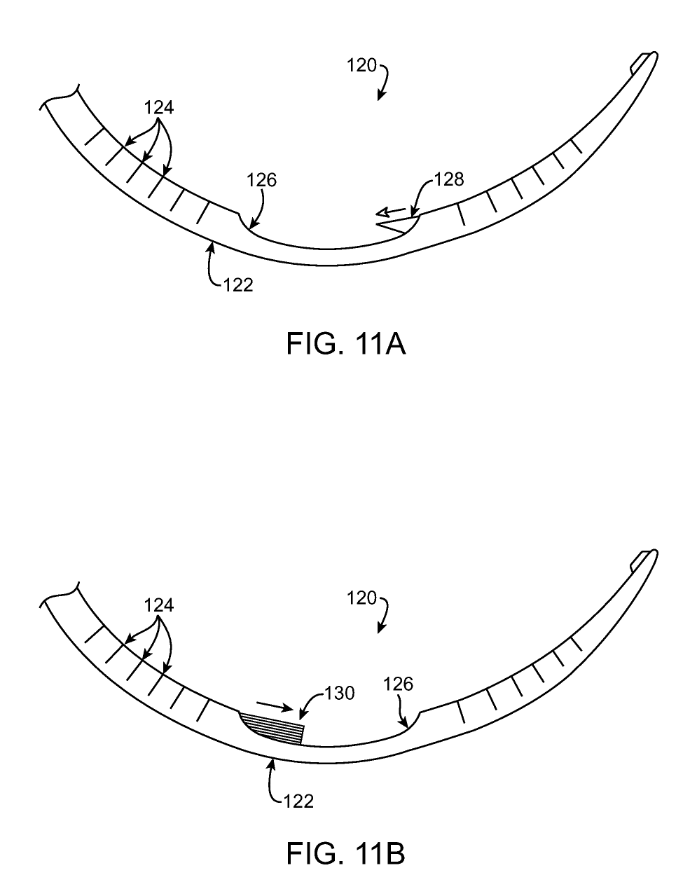

With reference now to FIGS. 11A and 11B, in one embodiment, a tissue cutter device 120 (only a portion of which is shown) may include a shaft 122 having multiple slits 124 for flexibility and a window 126, and multiple cutting members, which may be advanced into window 126 to cut tissue. In some embodiments, for example, it may be advantageous to have one or more cutting members for cutting soft tissue, such as ligament, and one or more cutting members for cutting hard tissue, such as bone. For example, in one embodiment, referring to FIG. 11A, a distal blade 128 may be advanced (hollow-tipped arrow) and used to cut soft tissue, such as ligament. Blade 128 may then optionally be retracted back into shaft 122, and (referring to FIG. 11B) a wire bundle cutting member 130 may be advanced (solid-tipped arrow) to cut bone. In one embodiment, for example, distal blade 128 may be used to cut tissue by manually moving shaft back and forth to caused blade 128 to slice tissue, while wires 130 may be reciprocated rapidly, such as by ultrasound power, to ablate or pulverize bone.

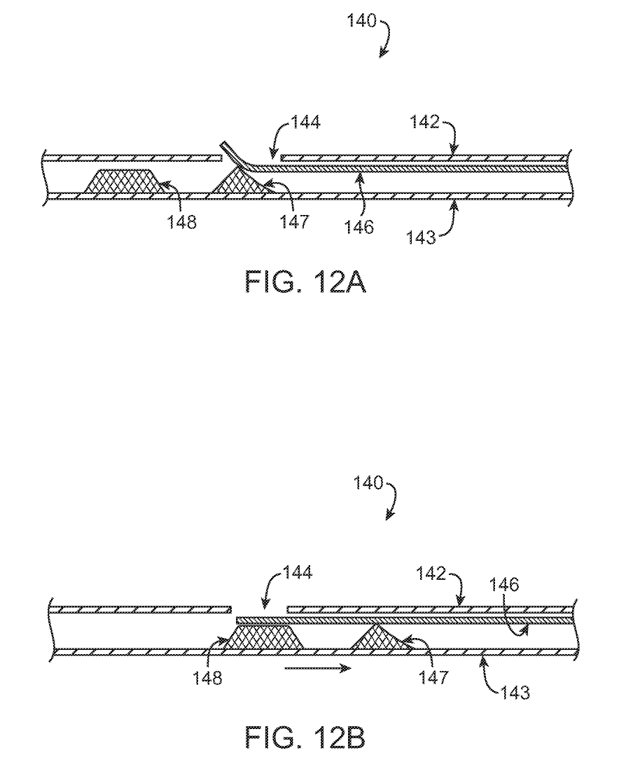

Referring to FIGS. 12A and 12B, in another alternative embodiment, a tissue cutter device 140 (only a portion of which is shown) may include a stationary shaft portion 142 having a window 144, a moveable shaft portion 143, a wire bundle 146, and a ramp 147 and plateau 148 coupled with an inner surface of moveable portion 143. When moveable portion 143 is placed in a first position, ramp 147 deflects a distal end of wire bundle 146 out of window 144 to facilitate tissue removal, such as of soft tissue, and to control the depth of tissue cut. Moveable portion 143 may be repositioned (FIG. 12B, hollow-tipped arrow) to bring ramp within stationary shaft 142, such that wire bundle 146 is not deflected out of window 144 but instead travels forward in a relatively straight direction over plateau 148. Reciprocating wire bundle 146 back and forth in a relatively straight path may be advantageous for cutting hard tissue, such as bone.

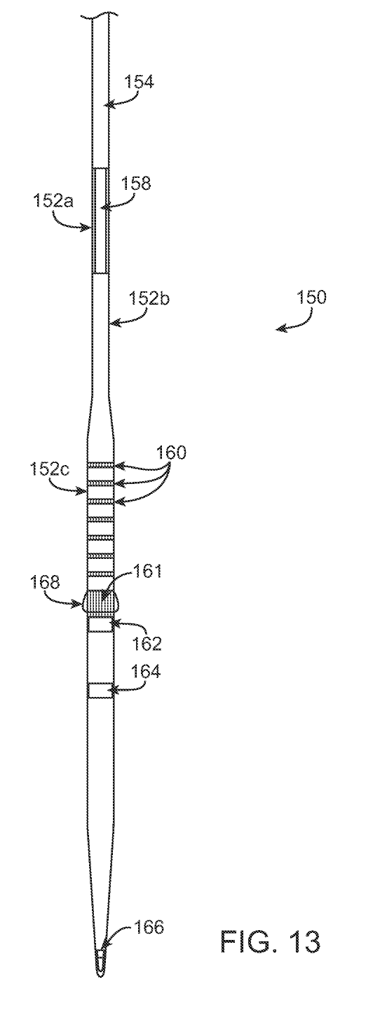

In an alternative embodiment, as shown in FIG. 13, a tissue cutter device 150 may be configured similarly to the embodiment shown in FIGS. 5A-5E but may further include a radiofrequency (RF) wire loop cutter 168. As in the earlier-described embodiment, cutter device 150 may include a movable shaft portion 154, a proximal stationary shaft portion 152a, a distal stationary shaft portion 152b, and a flexible shaft portion 152c having multiple slits 160 for enhanced flexibility. Device 150 may also include a wire bundle tube 158 into which a proximal end of a wire bundle 161 is secured, a proximal blade 162 coupled with the distal end of wire bundle 161, a distal blade 164, and a guidewire connector 166. In addition, in one embodiment, device 150 may further include RF wire loop 168, which may optionally be retractable into shaft 152c. RF energy may be applied to loop cutter 168, for example, for cutting soft tissue such as ligament. Blades 162, 164 may be used to cut additional soft tissue and/or to cut bone.

Wire loop 168 may comprise any suitable RF electrode, such as those commonly used and known in the electrosurgical arts, and may be powered by an internal or external RF generator, such as the RF generators provided by Gyrus Medical, Inc. (Maple Grove, Minn.). Any of a number of different ranges of radio frequency may be used, according to various embodiments. For example, some embodiments may use RF energy in a range of between about 70 hertz and about 5 megahertz. In some embodiments, the power range for RF energy may be between about 0.5 Watts and about 200 Watts. Additionally, in various embodiments, RF current may be delivered directly into conductive tissue or may be delivered to a conductive medium, such as saline or Lactated Ringers solution, which may in some embodiments be heated or vaporized or converted to plasma that in turn modifies target tissue. In various embodiments, wire loop 168 may be caused to extend out of a window of a shaft, expand, retract, translate and/or the like. One or more actuators (not shown) for manipulating and/or powering wire loop 168 will typically be part of device 150 and may either be coupled with, integrated with or separate from an actuator for reciprocating wire bundle 161.

The embodiment shown in FIG. 13 is only one example of how, in some embodiments, multi-wire tissue cutter device 150 may employ two or more different cutting modalities in the same device. For example, one tissue cutter device may include, in addition to a multi-wire bundle, any one or more of such tissue manipulation devices as a rongeur, a curette, a scalpel, a scissors, a forceps, a probe, a rasp, a file, an abrasive element, a plane, a rotary powered mechanical shaver, a reciprocating powered mechanical shaver, a powered mechanical burr, a laser, an ultrasound crystal a cryogenic probe, a pressurized water jet, a drug dispensing element, a needle, a needle electrode, or some combination thereof. In some embodiments, for example, it may be advantageous to have one or more tissue modifying members that stabilize target tissue, such as by grasping the tissue or using tissue restraints such as barbs, hooks, compressive members or the like. In one embodiment, soft tissue may be stabilized by applying a contained, low-temperature substance (for example, in the cryo-range of temperatures) that hardens the tissue, thus facilitating resection of the tissue by a blade, rasp or other device. In another embodiment, one or more stiffening substances or members may be applied to tissue, such as bioabsorbable rods.

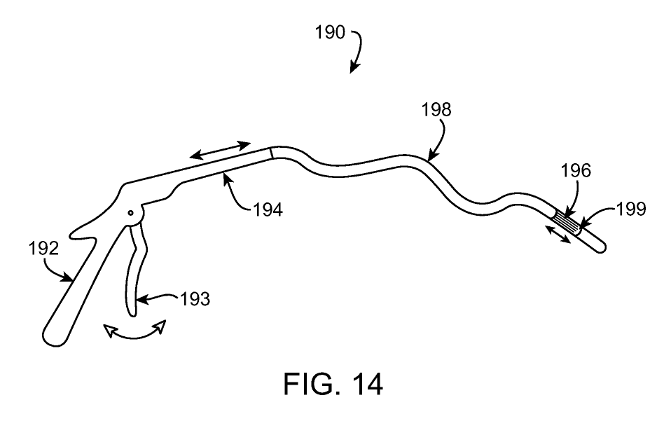

With reference now to FIG. 14, in another embodiment, a multi-wire tissue cutter device 190 may include a proximal handle 192 with an actuator 193, a rigid shaft portion 194 extending from handle 192, an elongate flexible shaft portion 198 extending from rigid shaft 194 and having a window 199, and a wire bundle 196 extending through flexible shaft 198 and into window 199. In various embodiments, rigid portion 194 and flexible portion 198 may have any desired lengths. When actuator 193 is squeezed and released (hollow-tipped, double-headed arrow), a driving mechanism in rigid shaft portion 194 reciprocates (solid-tipped, double-headed arrow), thus causing wire bundle 196 to reciprocate (open, double-tipped arrow) to cut or otherwise ablate tissue.

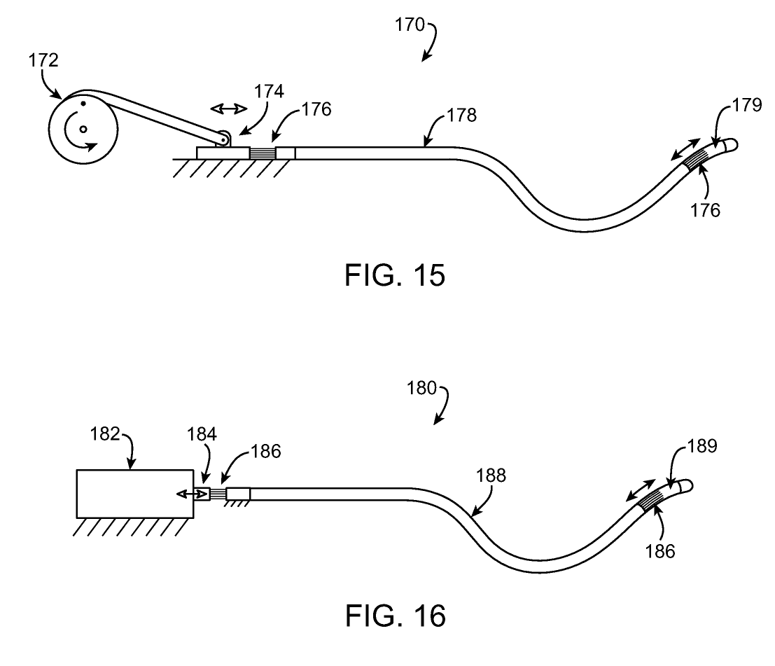

FIG. 15 shows another embodiment of a multi-wire tissue cutter device 170, including a motor 172, a drive shaft 174, an at least partly flexible shaft 178 having a window 179, and a wire bundle 176 slidably disposed within shaft 178 and extending into window 179 to cut tissue. Generally, motor 172 rotates about a central axis (solid-tipped arrow) to cause drive shaft 174 to reciprocate (hollow-tipped, double-headed arrow), thus moving wires back and forth through shaft 178. At least a proximal portion of shaft 178 remains stationary (diagonal lines), relative to drive shaft 174, so that wire bundle 176 moves through shaft.

In another embodiment, and with reference now to FIG. 16, a tissue cutter device 180 may include an ultrasound source 182, a drive shaft 184 coupled with source 182, a wire bundle 186 coupled with drive shaft 184, and an at least partly flexible shaft 188 with a window 189. In this embodiment, ultrasound source 182 and a proximal portion of shaft 188 (such as a proximal handle or the like) remain stationary, and drive shaft 184 reciprocates (hollow-tipped, double-headed arrow) to reciprocate wire bundle 186 through shaft 188. The distal end of wire bundle 186, reciprocated at ultrasonic frequencies, may be used to cut or ablate soft tissue and/or bone. In various alternative embodiments, other alternative mechanisms for driving a bundle of wires, such as gears, ribbons or belts, magnets, electrically powered, shape memory alloy, electro magnetic solenoids and/or the like, coupled to suitable actuators, may be used. In one alternative embodiment, for example, an hydraulic fluid may extend through a portion of shaft 188 to drive wire bundle 186.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

We claim:

1. A device for cutting tissue in a human body, the device comprising:
an elongate shaft having a proximal portion and a distal portion;
at least one translatable blade disposed along one side of the distal portion of the shaft, wherein the blade has a height greater than a height of a portion of the shaft immediately below the blade, and wherein a total height of the blade and the portion of the shaft immediately below the blade is less than a width of the portion of the shaft immediately below the blade; and
at least one actuator coupled with the at least one translatable blade and extending to the proximal portion of the shaft, wherein the actuator is configured to translate the blade to cut tissue.

2. A device as in claim 1, wherein the distal portion of the shaft is sized to pass into an epidural space and at least partway into an intervertebral foramen of a spine.

3. A device as in claim 2, further comprising one of a backstop and a stationary blade toward which the translatable blade moves to cut tissue, wherein an edge of the backstop or stationary blade is disposed at a blade opening distance from a cutting edge of the translatable blade.

4. A device as in claim 3, wherein the blade opening distance is between about 0.3 inches and about 0.35 inches, the height of the portion of the shaft immediately below the translatable blade is between about 0.025 inches and about 0.035 inches, the height of the translatable blade is between about 0.040 inches and about 0.060 inches, and the width of the portion of the shaft immediately below the blade is between about 0.165 and about 0.250 inches.

5. A device as in claim 1, wherein a ratio of the height of the translatable blade to the height of the portion of the shaft immediately below the blade is no less than 4/3.

6. A device as in claim 1, wherein a ratio of the total height of the translatable blade and the height of the portion of the shaft immediately below the blade to the width of the portion of the shaft immediately below the blade is no greater than ¾.

7. A device as in claim 1, further comprising a guidewire coupling member disposed on the distal portion of the shaft for coupling the shaft with a guidewire to pull the device into a desired position and/or to apply tensioning force to the device to urge the translatable blade against target tissue.

8. A device as in claim 1, wherein the at least one actuator comprises:
at least two flexible wires extending through a hollow lumen of the shaft to couple the actuator to the at least one translatable blade; and
a proximal actuation member coupled with the wires and the proximal portion of the shaft, wherein activating the actuation member advances the wires to advance the blade along the shaft.

9. A device as in claim 1, wherein the at least one actuator comprises:
at least one flexible wire extending through a hollow lumen of the shaft to couple the actuator to the at least one translatable blade; and
a proximal actuation member coupled with the wire(s) and the proximal portion of the shaft, wherein activating the actuation member retracts the wire(s) to retract the blade along the shaft.

10. A device as in claim 1, further comprising at least one chamber in or on the shaft for collecting cut tissue.

11. A device as in claim 1, wherein the shaft further comprises a flexible portion disposed between the proximal and distal portions, the device further comprising at least one shaft flexing actuator coupled with the proximal portion of the shaft and extending at least to the flexible portion of the shaft.

12. A system for cutting tissue in a human body, the system comprising:
a tissue cutting device, comprising:
an elongate shaft having a proximal portion and a distal portion;
at least one translatable blade disposed along one side of the distal portion of the shaft, wherein the blade has a height greater than a height of a portion of the shaft immediately below the blade, and wherein a total height of the blade and the portion of the shaft immediately below the blade is less than a width of the portion of the shaft immediately below the blade;
at least one actuator coupled with the at least one translatable blade and extending to the proximal portion of the shaft, wherein the actuator is configured to translate the blade to cut tissue; and
a guidewire coupling member disposed on the distal portion of the shaft for coupling the shaft with a guidewire to pull the device into a desired position and/or to apply tensioning force to the device to urge the translatable blade against target tissue; and
a guidewire configured to couple with the guidewire coupling member.

13. A system as in claim 12, wherein the distal portion of the shaft of the tissue cutting device is sized to pass into an epidural space and at least partway into an intervertebral foramen of a spine.

14. A system as in claim 13, wherein the tissue cutting device further comprises one of a backstop and a stationary blade toward which the translatable blade moves to cut tissue, wherein an edge of the backstop or stationary blade is disposed at a blade opening distance from a cutting edge of the translatable blade.

15. A system as in claim 14, wherein the blade opening distance is between about 0.3 inches and about 0.35 inches, the height of the portion of the shaft immediately below the translatable blade is between about 0.025 inches and about 0.035 inches, the height of the translatable blade is between about 0.040 inches and about 0.060 inches, and the width of the portion of the shaft immediately below the blade is between about 0.165 and about 0.250 inches.

16. A system as in claim 12, wherein a ratio of the height of the translatable blade to the height of the portion of the shaft immediately below the blade is no less than 4/3.

17. A system as in claim 12, wherein a ratio of the total height of the translatable blade and the height of the portion of the shaft immediately below the blade to the width of the portion of the shaft immediately below the blade is no greater than ¾.

18. A system as in claim 12, A device as in claim 1, wherein the at least one actuator of the tissue cutting device comprises:
at least two flexible wires extending through a hollow lumen of the shaft to couple the actuator to the at least one translatable blade; and
a proximal actuation member coupled with the wires and the proximal portion of the shaft, wherein activating the actuation member advances the wires to advance the blade along the shaft.

19. A system as in claim 12, wherein the at least one actuator of the tissue cutting device comprises:
at least one flexible wire extending through a hollow lumen of the shaft to couple the actuator to the at least one translatable blade; and
a proximal actuation member coupled with the wire(s) and the proximal portion of the shaft, wherein activating the actuation member retracts the wire(s) to retract the blade along the shaft.

20. A system as in claim 12, further comprising at least one chamber in or on the shaft of the device for collecting cut tissue.

21. A system as in claim 20, further comprising at least one of a suction device and an irrigation device removably couplable with the tissue cutting device to provide at least one of suction and irrigation to the chamber to remove the cut tissue from the device, and wherein the shaft of the tissue cutting device includes at least one lumen for at least one of suction and irrigation.

22. A system as in claim 12, wherein the shaft of the device further comprises a flexible portion disposed between the proximal and distal portions, the device further comprising at least one shaft flexing actuator coupled with the proximal portion of the shaft and extending at least to the flexible portion of the shaft.

23. A system as in claim 12, further comprising a guidewire handle for coupling with the guidewire outside the body to facilitate pulling the device into position and/or applying tensioning force.
